(19) United States
(12) Patent Application Publication
(10) Pub. No.: US 2021/0106828 A1
(43) Pub. Date: Apr. 15, 2021

(54) CHIP ASSEMBLY FOR IMPLANTATION INTO LIVING TISSUE

(71) Applicant: IRIDIUM MEDICAL TECHNOLOGY CO., LTD., Hsinchu (TW)

(72) Inventors: Long-sheng FAN, Hsinchu (TW); Yun-Ta YANG, Hsinchu (TW)

(21) Appl. No.: 17/065,323

(22) Filed: Oct. 7, 2020

Related U.S. Application Data
(60) Provisional application No. 62/913,305, filed on Oct. 10, 2019.

(51) Int. Cl. A61N 1/36; A61N 1/05; A61N 1/04

(52) U.S. Cl. CPC A61N 1/36046; A61N 1/0472; A61N 1/0543

(57) ABSTRACT

The invention provides a chip assembly for implantation into a living tissue comprising an electronic element and a biocompatible buffer material. The electronic element is defined to form a first contour, the first contour comprises at least one sharp edge exposed outside, and the buffer material covers the sharp edge and blocks the sharp edge to avoid damage to the living tissue.

### FIELD OF THE INVENTION

The present invention relates to a chip assembly, and particularly relates to an electronic element used for implantation into a living tissue.

### BACKGROUND OF THE INVENTION

Among the patients with vision deterioration, some patients choose to implant retinal prosthesis to improve their vision. At present, commercial retinal prosthesis is expensive and low in the pixel, and the improvement in the quality of life of the patients is limited. In view of this, many companies and academic research units have begun to actively invest in improving the retinal prosthesis microsystem.

For example, U.S. patent Ser. No. 10/760,961, U.S. Pat. Nos. 8,530,265, 8,954,156, 9,114,004, 9,155,881, 9,731,130, etc. disclose that the retinal prosthesis is a chip implanting in a living tissue. In some patents, the chip uses a semiconductor process to manufacture a microelectrode, a photosensor, and other circuits on a silicon substrate, even including a processor and a driver. It can be expected that the material of the chip is relatively hard; and in view of circuit design and complex manufacturing process, the appearance of the chip is difficult to be processed into an ideal shape. In summary, the chip is likely to cause tissue damage when implanted into the living tissue.

### SUMMARY OF THE INVENTION

The present invention relates to a chip assembly, and in particular to a chip assembly for implantation into a living tissue, which can reduce or avoid damage after the chip assembly, is implanted into the living tissue.

The present invention provides a chip assembly for implantation into a living tissue, comprising: an electronic element, which is defined to form a first contour, and the first contour comprises at least one sharp edge exposed outside; and a buffer material with biocompatibility, which covers the sharp edge and blocks the sharp edge to avoid damage to the living tissue.

The present invention also provides a retinal prosthesis assembly, comprising: a retinal prosthesis chip, which comprises a plurality of light sensing assemblies receiving light, a plurality of microelectrodes, and a circuit coupled to the light sensing assemblies and the microelectrodes. The circuit drives the microelectrodes to provide nerve cells at least one stimulus to perceive an image of the light captured by the light sensing assemblies, wherein the light sensing assemblies, the microelectrodes and the circuit are integrated in a semiconductor device, which comprises a silicon substrate carrying the microelectrodes; an encapsulation layer which at least partially covers the retinal prosthesis chip to protect the retinal prosthesis chip; and a buffer material with biocompatibility covering at least one sharp edge of the retinal prosthesis chip and blocking the sharp edge to avoid damage to the eyeball tissue.

### BRIEF DESCRIPTION OF THE DRAWINGS

FIG. 1 is a schematic diagram of the retinal prosthesis implanted in the eyeball.

FIG. 2 is a schematic diagram of a retinal prosthesis device according to an embodiment.

FIG. 3 is a schematic diagram of a retinal prosthesis chip according to an embodiment.

FIG. 4A and FIG. 4B are section schematic diagrams of the retinal prosthesis assembly according to an embodiment.

FIG. 5 is a section schematic diagram of the retinal prosthesis chip according to an embodiment.

FIG. 6A and FIG. 6B are schematic diagrams of a retinal prosthesis chip according to an embodiment.

FIG. 7A and FIG. 7B are schematic diagrams of the structure distribution of pixel units according to an embodiment.

FIG. 8 is a section schematic diagram of the retinal prosthesis chip according to an embodiment.

FIG. 9 is a section schematic diagram of the retinal prosthesis chip according to another embodiment.

FIG. 10 is a section schematic diagram of the retinal prosthesis chip according to another embodiment.

FIG. 11 is a section schematic diagram of the retinal prosthesis chip according to another embodiment.

FIG. 12 is a schematic diagram of an electrical connection portion implanted in a human body according to an embodiment.

FIG. 13 is a schematic diagram of an electrical connection portion according to an embodiment.

FIG. 14 is a section schematic diagram of FIG. 13.

FIG. 15 is a section schematic diagram of an electrical connection portion according to an embodiment.

FIG. 16 is a section schematic diagram of an electrical connection portion according to another embodiment.

FIG. 17 is a section schematic diagram of an electrical connection portion according to another embodiment.

FIG. 18 is a section schematic diagram of an electrical connection portion according to another embodiment.

FIG. 19 is a section schematic diagram of an electrical connection portion according to an embodiment.

### DETAILED DESCRIPTION OF THE PREFERRED EMBODIMENTS

First, the terms used in the description of various embodiments are only for the purpose of describing specific examples, and are not intended to be limiting. Unless the context clearly indicates otherwise, or does not deliberately limit the quantity of the assembly, the singular forms “a”, “an” and “the” used herein also include plural forms. On the other hand, the terms “comprising” and “including” are intended to be included, meaning that there may be additional assemblies other than the listed assemblies.

When an assembly is expressed as being “connected” or “coupled” to another assembly, the assembly can be connected or coupled to the other assembly directly or through an intermediate assembly; additionally, it should be understood that the description order of various embodiments should not be explained as implying that the operations or steps must be dependent on the order, and alternative embodiments may use the order different from the order described herein to perform the steps, operations, methods, etc.

The present invention provides an implantable chip assembly for implantation into a living tissue. The implantable chip assembly comprises an electronic element and a biocompatible buffer material. The electronic element is formed with a first contour, and the first contour comprises at least one exposed sharp edge. The biocompatible buffer material is formed with a second contour, and the second contour is composed of a plurality of flexible planes and a plurality of blunt edges connected between the flexible planes. In an embodiment, the first contour comprises a plurality of planes, and the sharp edge is formed between the planes.

As a buffer structure, the biocompatible buffer material can relieve the damage on the living tissue caused by physical compression, contact puncture or scratches generated by the hard material of the electronic element and the edges, corners on the appearance or other sharp structures. Further, any allergy or rejection situation of cell tissue to the implant is reduced. Therefore, the implantable chip assembly can be applied to various semiconductor devices that require long-term contact with human tissues, such as a subcutaneous chip, a drug release chip, a nerve stimulation chip, an artificial electronic ear, an artificial retina and other biomedical chips.

The following takes the retinal prosthesis as an example to illustrate the specific structure of the implantable chip assembly. Age-related macular degeneration (AN/ID) and retinitis pigmentosa (RP) are the main causes of blindness. The patients lose their ability of producing visual signals due to the degeneration of photoreceptor cells in the retina. However, considering that the bipolar cells and ganglion cells on the retina of the patient still retain partial functions, the retinal prosthesis can be implanted to generate electrical stimulation signals to stimulate these nerve cells to produce visual signals, so the degraded photoreceptor cells can be replaced.

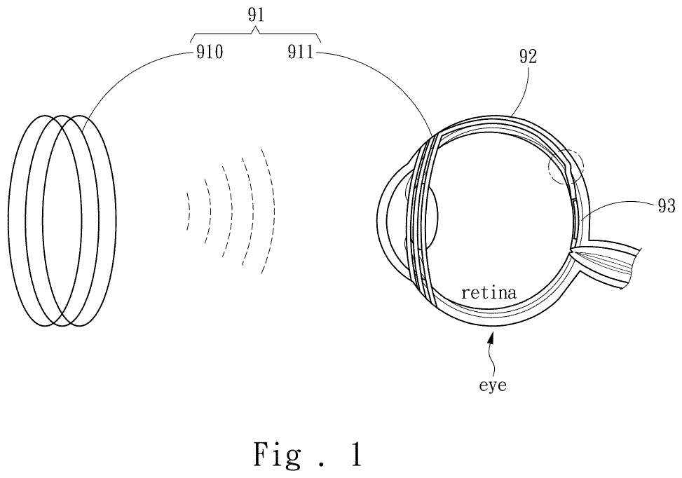

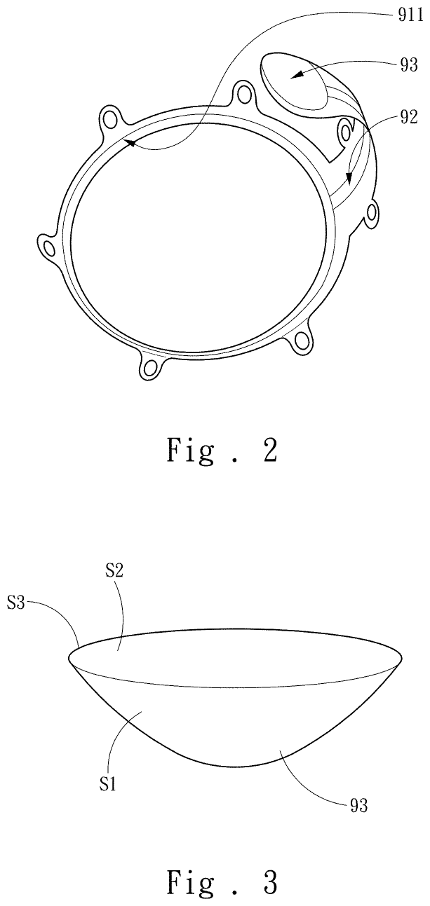

FIG. 1 is a schematic diagram of the retinal prosthesis implanted in the eyeball; and FIG. 2 is a schematic diagram of a retinal prosthesis device according to one embodiment. The retinal prosthesis device in FIG. 1 and FIG. 2 comprises a set of induction coils 91 providing a power source, an electrical connection portion 92 transmitting electronic signals, and a retinal prosthesis chip 93 stimulating nerve cells. In detail, the set of induction coils 91 comprises an external induction coil 910 and an internal induction coil 911 inductively coupled to the external induction coil 910. The retinal prosthesis chip 93 is composed of a plurality of pixel units. Each pixel unit comprises a photosensor, a signal processing unit, and a stimulation electrode. The photosensor generates a sensing signal after receiving an incident light, the signal processing unit receives and processes the sensing signal to generate an electrical stimulation waveform, and the stimulation electrode correspondingly generates a stimulation current to stimulate a retinal cell after receiving the electrical stimulation waveform. Further, the retinal prosthesis chip 93 is selected from an epi-retinal implant and a sub-retinal implant according to the implantation position.

Referring to FIG. 3 for the three-dimensional schematic diagram of the retinal prosthesis chip 93, the retinal prosthesis chip 93 is made of a single flexible element, and takes a standard or fine-tuned CMOS technology or CMOS image sensing (CIS) technology to integrate pixel units into an electrode array on a silicon chip. Each pixel unit comprises a stimulation electrode, a light sensing assembly, and a processor and a drive circuit (not shown in the figures). The light sensing assembly can be a PN junction diode made by improved CMOS technology; or the light sensing assembly can be an anti-reflection coating layer with an appropriate doping contour made by CIS technology. In addition, the silicon chip is a laminated structure with the protruded stimulation electrode, wherein the laminated structure includes a polymer barrier layer with metal/dielectric layer and silicon covering the assembly together. The stimulation electrode can protrude with a protruding end close to a target nerve cell to contact the nerve cell. Further, the thickness of the chip is very thin enough to bend, for example, the radius of the chip is about 3 mm, and it can be bent to about 90 microns from the center of the chip to the edge of the chip so as to form a two-dimensional spherical-like curved surface.

In general, the retinal prosthesis chip usually comprises a plurality of electronic elements, and the plurality of electronic elements is usually made from hard materials and has sharp edges. Therefore, it is known that the retinal prosthesis chip is prone to generate allergy or rejection reaction with human tissues after implanted, and is also prone to damage the human tissues. Although there are a multilayer structure usually arranged at the periphery of the retinal prosthesis chip based on the consideration of the biocompatibility and encapsulation, the multilayer structure must still have sufficient hardness to maintain the structure of the retinal prosthesis and provide sufficient supporting force. For the soft tissue in the eye, the hardness of the multilayer structure is too high to cause friction with the tissue cells during implantation, which causes the wear of the tissue cells and is not beneficial to long-term implantation.

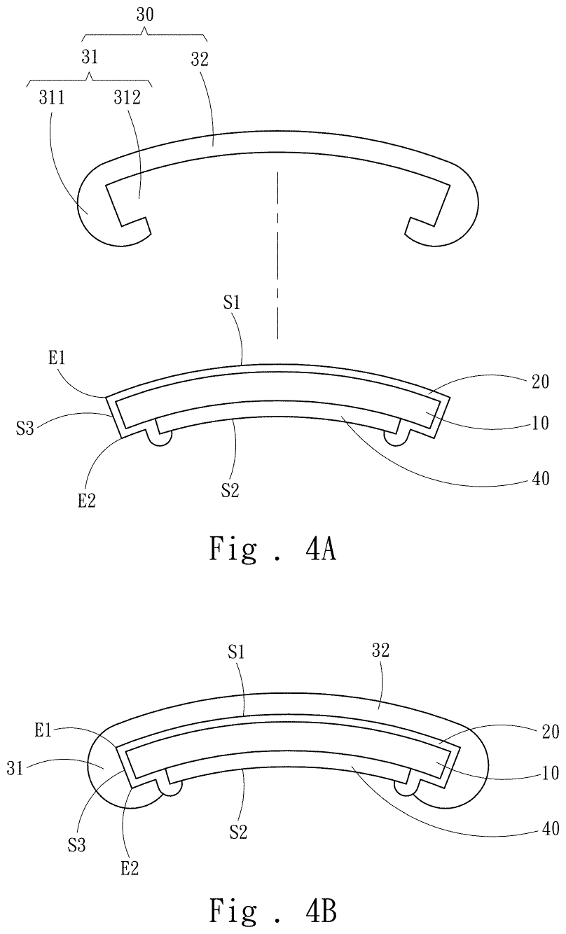

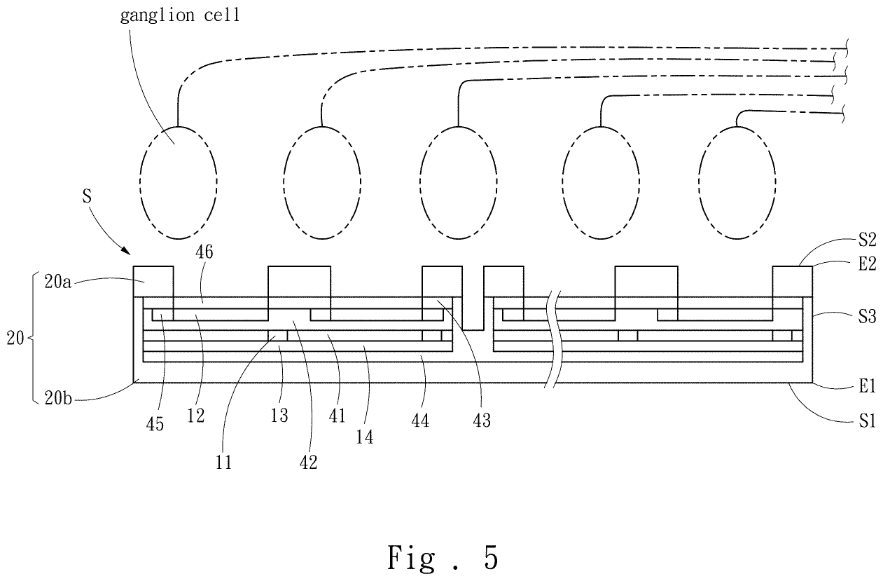

FIG. 4A and FIG. 4B show the retinal prosthesis assembly according to an embodiment of the present invention. The retinal prosthesis assembly comprises a retinal prosthesis chip 10, an encapsulation layer 20, and a buffer material 30 with biocompatibility. Referring to FIG. 5, the retinal prosthesis chip 10 comprises a plurality of light sensing assemblies 11, a plurality of microelectrodes 12 and a circuit 13. The encapsulation layer 20 comprises a first package layer 20a and a second package layer 20b. The plurality of light sensing assemblies 11 receives the light; the circuit 13 is coupled to the plurality of light sensing assemblies 11 and the plurality of microelectrodes 12; and the circuit 13 drives the plurality of microelectrodes 12 to provide at least one stimulus to nerve cells and to perceive an image of the light captured by the plurality of light sensing assemblies 11. The plurality of light sensing assemblies 11, the plurality of microelectrodes 12, and the circuit 13 are integrated in one semiconductor device S. The semiconductor device S is a multilayer structure, which comprises an upper surface S1, a lower surface S2, and a periphery S3.

The semiconductor device S comprises a silicon substrate 14; and the circuit 13 is formed over the silicon substrate 14. In this embodiment, the semiconductor device S comprises a plurality of pixel units 40 formed on the silicon substrate 14. Each pixel unit 40 comprises the light sensing assembly 11, the microelectrode 12, and a signal processing and drive unit 41. Further, each pixel unit 40 comprises an intermediate layer 42, a first barrier layer 43, a second barrier layer 44, a guard ring 45, and a conductive layer 46. The intermediate layer 42 is arranged among the microelectrodes 12, the light sensing assemblies 11, and the signal processing and drive unit 41. The intermediate layer 42 may be an oxide layer, such as silicon dioxide (SiO₂). The encapsulation layer 20 at least partially covers the retinal prosthesis chip 10 to protect the retinal prosthesis chip 10. Specifically, the encapsulation layer 20 is made from a flexible material. The buffer material 30 covers at least one sharp edge of the retinal prosthesis chip 10 and blocks the sharp edge to avoid damage to the eyeball tissue. Referring to FIG. 4A, in this embodiment, the buffer material 30 comprises a peripheral buffer element 31 and an intermediate buffer element 32. The peripheral buffer element 31 comprises a ring-shaped body 311 and a ring-shaped notch 312 annularly arranged in the ring-shaped body 311. The buffer material 30 can be directly formed with the shape as shown in FIG. 4A when being manufactured, or utilize the flexibility to deform into the shape as shown in FIG. 4A.

In an embodiment, the buffer material 30 is selected from polyimide, polydimethylsiloxane (PDMS), parylene, liquid crystal polymer and other biocompatible materials. Further, in different embodiments, the buffer material 30 may be formed as an integral structure with elasticity. After being stretched, the buffer material 30 is sleeved to the edge of the retinal prosthesis chip 10; and the buffer material 30 may also include a clamping structure to be fastened to the retinal prosthesis chip 10 in a buckling manner; or the buffer material 30 may also include an adhesive layer to be fixed on the retinal prosthesis chip 10.

As to the application of the retinal prosthesis, the electrical stimulation is transmitted to nerve cells through the electrode array, and thus a neuron-to-electrode distance between the electrode array and nerve cells needs to be required. Therefore, the appearance structure of the retinal prosthesis chip 10 and the microelectrodes 12 needs to be considered accordingly. Besides, if it applied to implanting in the living tissues other than retina, similar requirements will also be raised. Here, only the implantation of the retina is taken as an example.

Further, the retinal prosthesis chip 10 is bent to a curvature conforming to the shape of the human eyeball. In an embodiment, the silicon substrate 14 is thinned to have a thickness that can be bent to conform to the shape of the human eyeball, as shown in FIG. 5. For example, the silicon substrate 14 is thinned enough so that the semiconductor device S can be bent 90 microns from the center to the edge, or bent to a radius of curvature less than 12 mm, so as to conform to the shape of the human eyeball within the limit of the material structure. In an example, the thickness of the silicon substrate 14 is approximately between 40 microns and 60 microns.

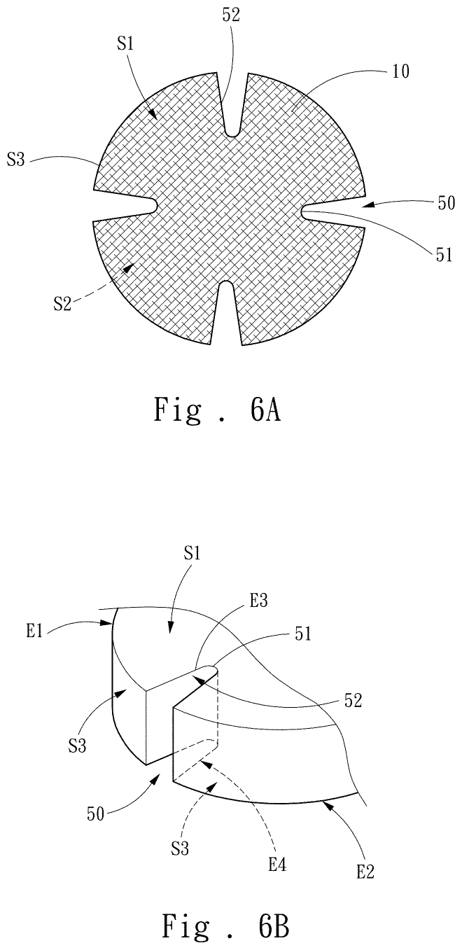

Referring to FIG. 6A and FIG. 6B, or in other embodiments, a plurality of cutout channels 50 is made on the retinal prosthesis chip 10, and thus the plurality of cutout channels 50 can reduce the deformation stress of the retinal prosthesis chip 10 so as to increase the allowable deformation angle of the retinal prosthesis chip 10. The plurality of cutout channels 50 longitudinally passes through the upper surface S1 and the lower surface S2 of the multilayer structure of the semiconductor device S of the retinal prosthesis chip 10, and extends inwardly from the periphery S3 (referring to FIG. 3). The plurality of cutout channels 50 is slot-shaped, and preferably arranged in a symmetrical radial shape. In addition, an inward end 51 of the cutout channels 50 can be rounded during manufacturing to achieve the effect of stress relief. In a bending state, the retinal prosthesis chip 10 forms a deformation that adjacent two sides of the cutout channels 50 approach each other gradually, and even contact each other (namely, the cutout channels 50 are closed) to produce a deformation, thereby allowing that the retinal prosthesis chip 10 is curved to conform to the shape of the human eyeball. Further, in one embodiment, the deformation is maintained by a fixing assembly, which is sleeved on a surrounding area of the semiconductor device S; or, the parts of the retinal prosthesis chip 10 adjacent to the two sides of the cutout channels 50 can be joined, such as welding, so that the bending state is maintained.

A first edge E1 is defined between the upper surface S1 and the periphery S3, a second edge E2 is defined between the lower surface S2 and the periphery S3, and side walls 52 of the cutout channels 50 together with the upper surface S1 and the lower surface S2 separately define a third edge E3 and a fourth edge E4. The sharp edge is formed on at least one of the first edge E1, the second edge E2, the third edge E3, and the fourth edge E4.

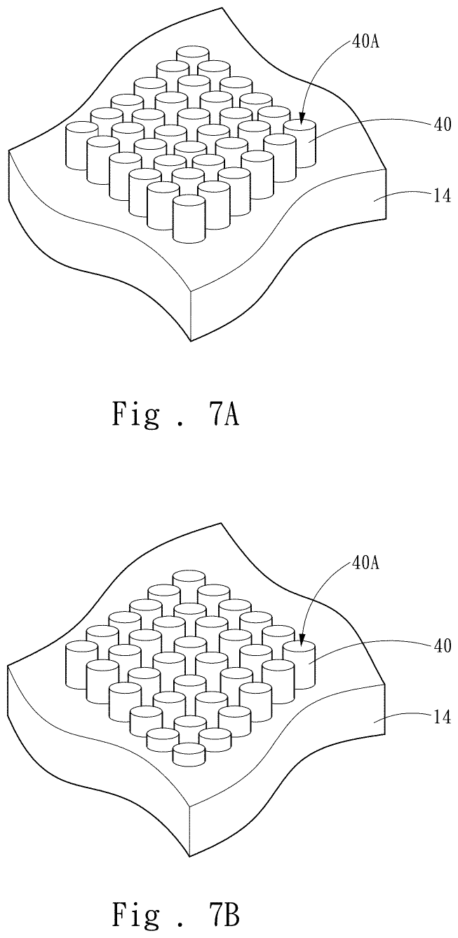

In the above embodiments, in an unbending state, protrude ends 40A of the pixel units 40 or sharp ends of the microelectrodes (not shown in figures) are distributed based on an imaginary plane, as shown in FIG. 7A; and in the bending state, the protrude ends 40A of the pixel units 40 (the sharp ends of the microelectrodes) are distributed in a quasi-spherical geometry based on the shape of the human eyeball.

In another embodiment, the silicon substrate 14 is not bent, but a flat substrate is adopted, and the height of the pixel units 40 (the microelectrodes) is manufactured to be non-equal height. Namely, the protrude ends 40A of the pixel units 40 (the sharp ends of the microelectrodes) are directly distributed in a quasi-spherical geometry to conform to the shape of the human eyeball, so that the neuron-to-electrode distance can be achieved between the pixel units 40 (the microelectrode) and the nerve cells without bending the device, as shown in FIG. 7B.

Different embodiments of the buffer material 30 covering the retinal prosthesis chip 10 are described below. For the convenience of description, the retinal prosthesis chip 10 covered with the encapsulation layer 20 is described as a retinal prosthesis device 10A as follows.

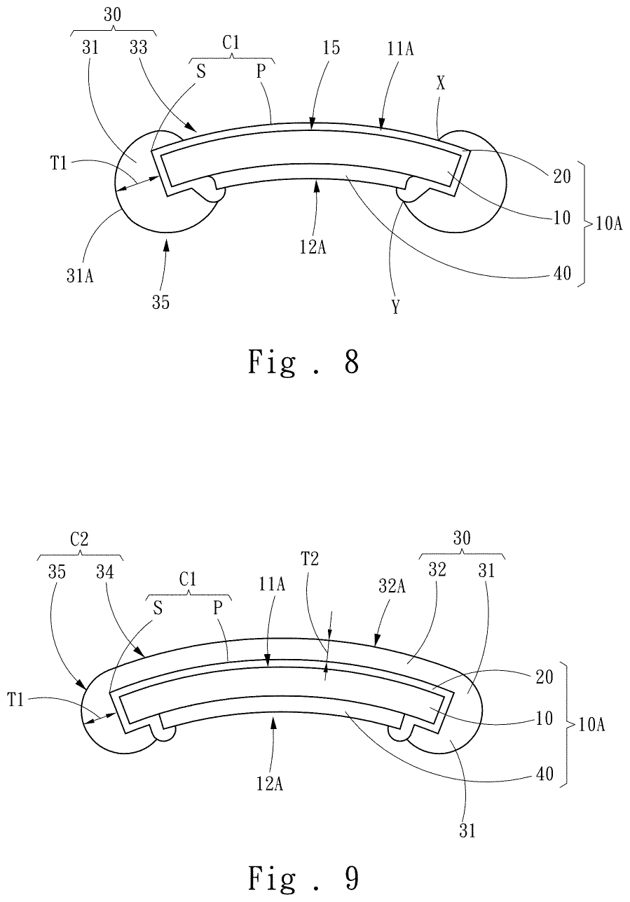

According to an embodiment of the present invention, taking the contour of the retinal prosthesis chip 10 as an example, the retinal prosthesis chip 10 comprises a plurality of flat surfaces, a plurality of curved surfaces, or a combination of a plurality of flat surfaces and curved surfaces. As illustrated in FIG. 8, the encapsulation layer 20 is defined to form a first contour C1 along an outer surface 15 of the retinal prosthesis chip 10, and the first contour C1 comprises a plurality of planes P, which may be flat or curved surfaces, and a plurality of sharp edges S formed among the planes P. From the perspective of mechanics of materials, the whole retinal prosthesis chip 10 can be regarded as a hard composite material, and the sharp edge in the present invention is defined as an edge, corner or protruding structure that can cause damage to the living tissue, not excluding the non-sharp appearance that can cause damage to the living tissue. The encapsulation layer 20 is a biocompatible encapsulation layer; and the material thereof can be selected according to the ISO 10993 standard. Since the thickness of the encapsulation layer 20 is thin, usually between several microns and 10 microns, even if the material is a biocompatible material, the encapsulation layer 20 does not have much influence on the rigidity of the retinal prosthesis device 10A.

In an embodiment, the hardness of the buffer material 30 is less than that of the retinal prosthesis chip 10, and is preferably an elastomer or a soft material, which can withstand high elastic deformation. For example, the buffer material 30 comprises polyimide, polydimethylsiloxane (PDMS), parylene, liquid crystal polymer or any combination of the above materials.

In an embodiment, as shown in FIG. 8, the buffer material 30 comprises a peripheral buffer element 31 and a hollow structure 33. The peripheral buffer element 31 is formed with a ring shape which surrounds the outer edge of the retinal prosthesis device 10A, and the retinal prosthesis device 10A comprises an upper surface 11A and a lower surface 12A. A plurality of microelectrodes (not shown in figures) is formed on the side close to the lower surface 12A, and the encapsulation layer 20 covers a side close to the upper surface 11A rather than covering a side close to the lower surface 12A to expose the microelectrodes. The peripheral buffer element 31 surrounds the retinal prosthesis device 10A from an edge Y of the lower surface 12A to an edge X of the upper surface 11A to form a continuous structure. A first thickness T1 is defined from the surroundings of the peripheral buffer element 31 and the retinal prosthesis device 10A to an outer surface 31A of the peripheral buffer element 31, and the first thickness T1 is ranged between 1 μm and 100 μm wherein the first thickness T1 is a variable value. In detail, the first thickness T1 gradually increases to a maximum value from a first end close to the upper surface 11A; and then gradually decreases from the maximum value to a second end close to the lower surface 12A.

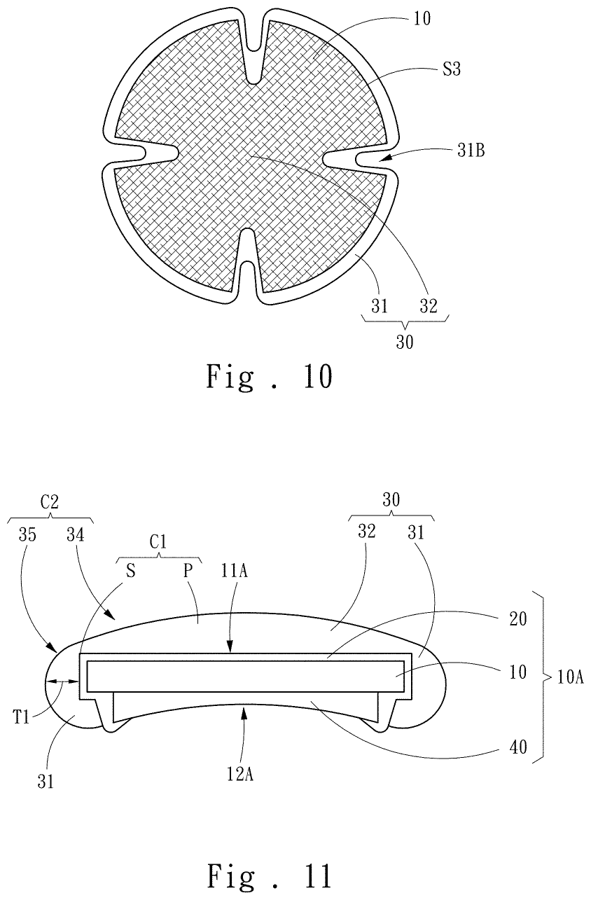

In another embodiment, as shown in FIG. 9, the buffer material 30 comprises a peripheral buffer element 31 and an intermediate buffer element 32. The intermediate buffer element 32 covers the upper surface 11A, and a second thickness T2 is defined from the surroundings of the intermediate buffer element 32 and the retinal prosthesis device 10A to an outer surface 32A of the intermediate buffer element 32, wherein the first thickness T2 is ranged between 1 μm and 100 μm.

The buffer material 30 is defined to form a second contour C2. In the embodiment of FIG. 9, the second contour C2 is composed of at least one flexible plane 34 and a plurality of blunt edges 35 joined with the flexible plane 34. Or, in other embodiments, the second contour C2 is only composed of a plurality of blunt edge 35. Besides, viewing from the section thereof, the peripheral buffer element 31 of the buffer material 30 can be regarded as a bump extending toward the outside of the retinal prosthesis device 10A, and the cross section view of the bump has a shape similar to a partial circle.

After the retinal prosthesis device 10A is implanted, the microelectrodes contact the retina of the human body, and accordingly the retina generates a pushing pressure to the retinal prosthesis device 10A. Thus, the arrangement of the buffer material 30 can relieve the problem that the retina or other tissues are hurt by the structure of the retinal prosthesis device 10A due to said pushing pressure. The above is only an example. In actual use, the configuration structure of the buffer material 30 is selected according to the implanted position of the implantable chip assembly, so that the buffer material 30 can fit the human tissue and avoid the damage of the electronic element to the human tissue.

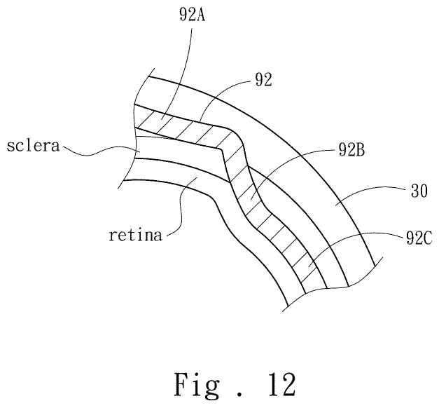

Further, if the retinal prosthesis chip 10 with the structure of FIG. 6A and FIG. 6B is adopted, the structure of the buffer material 30 is as shown in FIG. 10. The buffer material 30 comprises a peripheral buffer element 31 and an intermediate buffer element 32. The peripheral buffer element 31 covers the retinal prosthesis chip 10 along the periphery S3, and the peripheral buffer element 31 further comprises a groove 31B corresponding to the position of the cutout channel 50. In addition, if the retinal prosthesis chip 10 with the structure of FIG. 7 is adopted, the structure of the buffer material 30 is as shown in FIG. 11 accordingly.

In another embodiment, the electronic element is the electrical connecting portion 92 in FIG. 1, wherein FIG. 12 is a partial enlarged schematic diagram of the electrical connecting portion 92 in FIG. 1. It shows that the electrical connecting portion 92 comprises a first region 92A, a second region 92B and a third region 92C, the first region 92A extends therein from one end of the internal induction coil 911 along the area outside the sclera, the second region 92B extends the retina by passing through the sclera from outside the sclera, and the third region 92C extends along the retina. In one embodiment, the first region 92A, the second region 92B and the third region 92C of the electrical connecting portion 92 are all covered by the buffer material 30. In another embodiment, due to relatively fragile and sensitive retina tissue, only the second region 92B and the third region 92C of the electrical connecting portion 92 are covered by the buffer material 30.

Referring to FIG. 13, it shows a schematic diagram of the electrical connecting portion 92 covered by the buffer material 30. The electrical connecting portion 92 comprises a plurality of wires 920 embedded in a biocompatible layer 921, and the two ends of the wires 920 are electrically connected with the internal induction coil 911 and the retinal prosthesis chip 93 respectively. Further, the electrical connecting portion 92 is an extended strip element. The biocompatible layer 921 comprises an upper surface 921A, a lower surface 921B, a first side 921C and a second side 921D, wherein the upper surface 921A and the lower surface 921B respectively form a sharp edge S with the first side 921C and the second side 921D.

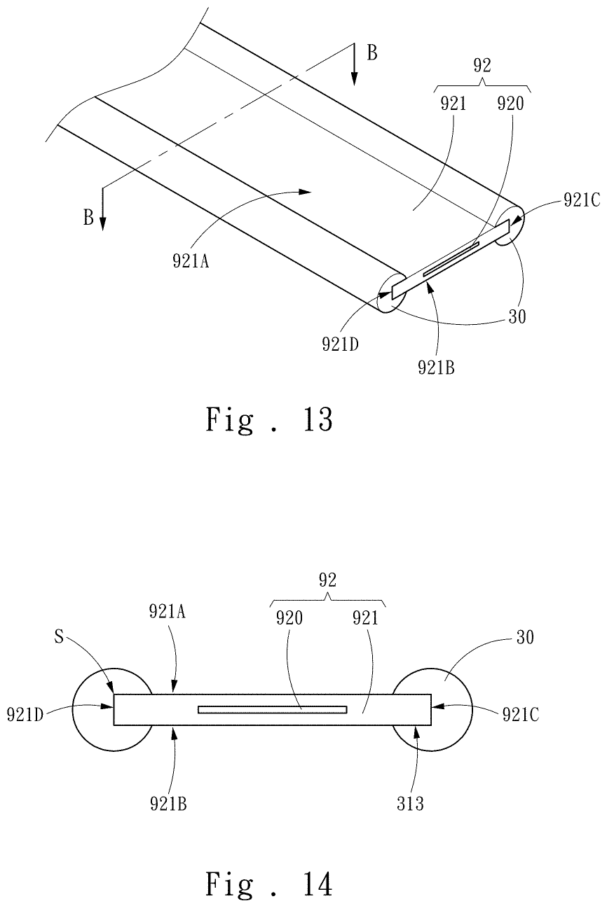

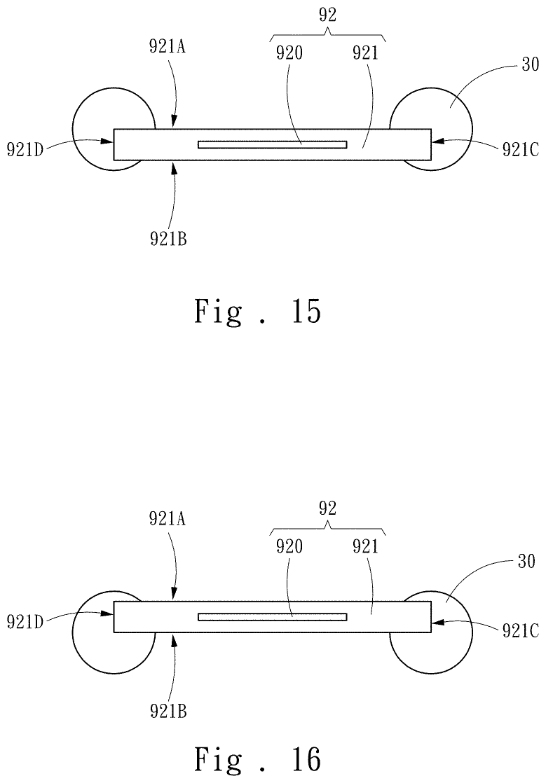

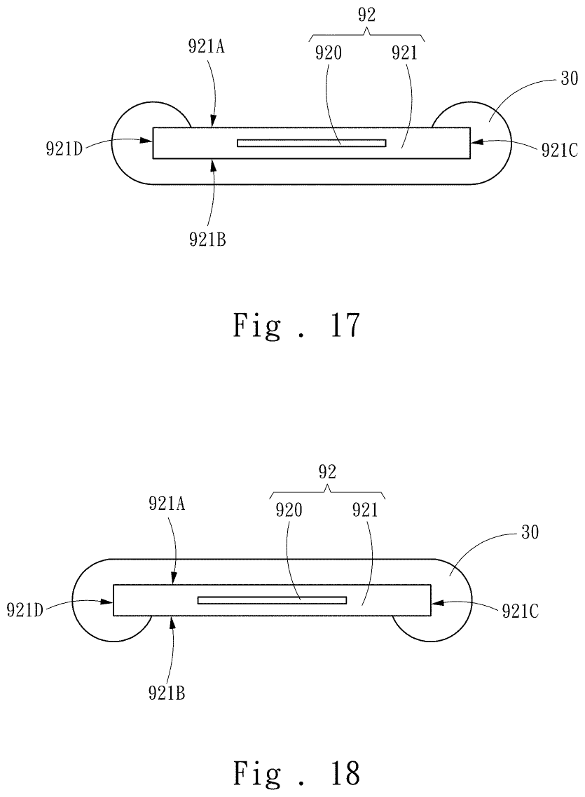

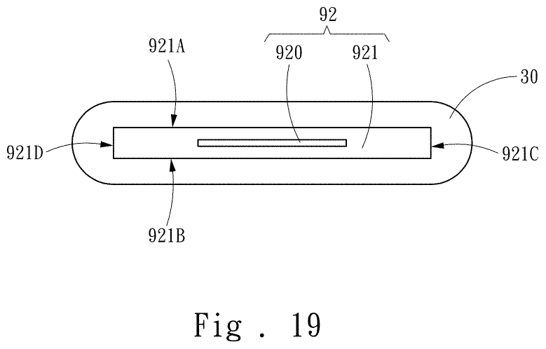

FIG. 14, FIG. 15, FIG. 16, FIG. 17, FIG. 18, and FIG. 19 are the section schematic diagrams of different embodiments in FIG. 13 along line B-B. In the embodiments of FIG. 14, FIG. 15, and FIG. 16, the buffer material 30 covers an upper sharp edge S from the edge of the upper surface 921A, and covers a lower sharp edge S along the first side 921C (or the second side 921D) to extend to the edge of the lower surface 921B, so that the middle parts of the upper surface 921A and the lower surface 921B of the electrical connecting portion 92 are exposed. The buffer material 30 is a cylinder with a transverse notch 313 when viewed from the section, and the difference between the embodiments of FIG. 14, FIG. 15, and FIG. 16 is that the transverse notch 313 is different relatively to the axis position of the cylinder.

In the embodiment of FIG. 17, the buffer material 30 extends from one side of the lower surface 921B to the other side to cover the lower surface 921B; in the embodiment of FIG. 18, the buffer material 30 extends from the one side of the upper surface 921A to the other side to cover the upper surface 921A; in the embodiment of FIG. 19, the buffer material 30 extends from one side of the lower surface 921B to the other side to cover the lower surface 921B, and also extends from one side of the upper surface 921A to the other side to cover the upper surface 921A.

In another embodiment, it provides a method of implanting a retinal prosthesis assembly in an epiretinal region of an eye, comprising the following steps of providing a first incision in sclera; providing a second incision in chorioidea; providing a third incision in retinal; inserting a guide element into the first incision, the second incision and the third incision to reach a position in the epiretinal region by sliding, wherein the guide element is provided with a guide surface; and introducing the retinal prosthesis assembly into the position of the epiretinal region along the guide surface of the guide element. In detail, the retinal prosthesis assembly comprises a retinal prosthesis chip; a biocompatible layer covering the retinal prosthesis chip to protect the retinal prosthesis chip; and a biocompatible buffer material, which covers at least one sharp edge of the retinal prosthesis chip and blocks the sharp edge to avoid damage to the eyeball tissue.

In the present invention, the buffer material 30 is not limited to a flat shape, and can be adjusted according to the specific structure of the human tissue, so that the buffer material 30 has an uneven surface to closely fit with the irregular tissue surface of the human body, and it is possible to improve the transmission efficiency of the signal and reduce the problem of assembly dropping thereof. The above is only an example. In actual use, the configuration structure of the buffer member is selected according to the implanted position of the implantable chip assembly, so that the buffer member can fit with the human tissue to reduce the gap between the electronic element and the buffer material, so as to increase the implantation stability and service life of the retinal prosthesis.

In summary, the implantable chip assembly of the present invention includes a buffer material coated on the electronic element such as the retina chip or the electrical connection portion. The buffer member is made from a biocompatible material, and the hardness of the buffer member is much less than that of the biocompatible encapsulation layer on the electronic element. After the implantable chip is implanted into the human body, the buffer member contacts and is fixed with tissue cells of the human body, so that the electronic element and the tissue cells are separated from each other to reduce side effects such as allergies, rejection or abrasion, further improve the treatment effect and prolong the service life of the implantable electronic chip. The buffer member has a plurality of different forms can be matched with the electronic element with various types and different structures for use. The buffer member can fit with surface of the tissue to be implanted, fix the electronic element, and achieve an optimal treatment effect.

What is claimed is:

1. A retinal prosthesis assembly, comprising:
a retinal prosthesis chip, comprising:
a plurality of light sensing assemblies, receiving a light;
a plurality of microelectrodes; and
a circuit, coupled to the plurality of light sensing assemblies and the plurality of microelectrodes, the circuit driving the plurality of microelectrodes to provide at least one stimulus to nerve cells and to perceive an image of the light captured by the plurality of light sensing assemblies;
wherein the plurality of light sensing assemblies, the plurality of microelectrodes and the circuit are integrated in one semiconductor device, and the semiconductor device comprises a silicon substrate carrying the plurality of microelectrodes;
an encapsulation layer, at least partially covering the retinal prosthesis chip to protect the retinal prosthesis chip; and
a buffer material with biocompatibility, covering at least one sharp edge of the retinal prosthesis chip and blocking the sharp edge to avoid damage to an eyeball tissue.

2. The retinal prosthesis assembly according to claim 1, wherein the silicon substrate is a flat substrate.

3. The retinal prosthesis assembly according to claim 2, wherein the retinal prosthesis chip is bent to conform to a shape of a human eyeball.

4. The retinal prosthesis assembly according to claim 3, wherein the silicon substrate is thinned to have a thickness that can be bent to conform to the shape of the human eyeball.

5. The retinal prosthesis assembly according to claim 3, wherein in an unbending state, sharp ends of the microelectrodes are distributed based on an imaginary plane, and wherein in a bending state, sharp ends of the microelectrodes are distributed in a quasi-spherical geometry based on the shape of the human eyeball.

6. The retinal prosthesis assembly according to claim 2, wherein in an unbending state, sharp ends of the microelectrodes are distributed in a quasi-spherical geometry based on a shape of a human eyeball.

7. The retinal prosthesis assembly according to claim 1, wherein the encapsulation layer is defined to form a first contour along an outer surface of the retinal prosthesis chip, and the first contour comprises a plurality of planes, and the sharp edge is formed among the planes.

8. The retinal prosthesis assembly according to claim 7, wherein the buffer material is defined to form a second contour, and the second contour is composed of at least one flexible plane and a plurality of blunt edges joined to the flexible plane.

9. The retinal prosthesis assembly according to claim 1, wherein the semiconductor device comprises a multilayer structure, and the multilayer structure comprises an upper surface, a lower surface and a periphery, and wherein a plurality of cutout channels pass through the upper surface and the lower surface longitudinally and are inwardly formed on the periphery of the multilayer structure, and the semiconductor device is bent into a shape conforming to a human eyeball through the plurality of cutout channels.

10. The retinal prosthesis assembly according to claim 9, wherein in a bending state, the semiconductor device forms a deformation that adjacent two sides of one of the cutout channels approach each other, and the deformation is maintained by a fixing assembly, which is sleeved on a surrounding area of the semiconductor device.

11. The retinal prosthesis assembly according to claim 9, wherein a first edge is defined between the upper surface and the periphery, a second edge is defined between the lower surface and the periphery, side walls of the cutout channels together with the upper surface and the lower surface separately define a third edge and a fourth edge, and the sharp edge is formed on at least any one of the first edge, the second edge, the third edge, and the fourth edge.

12. The retinal prosthesis assembly according to claim 1, wherein the buffer material comprises polyimide, polydimethylsiloxane (PDMS), parylene or the any combination of the above materials.

13. The retinal prosthesis assembly according to claim 1, wherein the buffer material includes a thickness greater than that of the encapsulation layer.

14. A chip assembly for implantation into a living tissue, comprising:
an electronic element, defined to form a first contour, the first contour comprising at least one sharp edge exposed outside; and
a buffer material with biocompatibility, the buffer material covering the sharp edge and blocking the sharp edge to avoid damage to the living tissue.

15. The chip assembly according to claim 14, wherein a hardness of the buffer material is less than a hardness of the electronic element.

16. The chip assembly according to claim 14, wherein the first contour comprises a plurality of planes, and the sharp edge is formed among the plurality of planes.

17. The chip assembly according to claim 14, wherein the buffer material is defined to form a second contour, and the second contour is composed of at least one flexible plane and a plurality of blunt edges joined to the flexible plane.

18. The chip assembly according to claim 14, wherein the buffer material comprises polyimide, polydimethylsiloxane (PDMS), parylene or the any combination of the above materials.
